(19) United States
(12) Patent Application Publication
(10) Pub. No.: US 2016/0333286 A1
(43) Pub. Date: Nov. 17, 2016

(54) FUEL REFORMING DEVICE

(71) Applicant: HONDA MOTOR CO., LTD., Tokyo (JP)

(72) Inventors: Tomohide KUDO, Wako (JP); Kohtaro HASHIMOTO, Wako (JP)

(73) Assignee: HONDA MOTOR CO., LTD., Tokyo (JP)

(21) Appl. No.: 15/084,480

(22) Filed: Mar. 30, 2016

(30) Foreign Application Priority Data
May 12, 2015 (JP) 2015-097541

(51) Int. Cl. C10L 10/10; C07C 29/50; C10L 1/18

(52) U.S. Cl. CPC C10L 10/10; C10L 1/18; C07C 29/50; C10L 1/1822

(57) ABSTRACT

A fuel reforming device includes a reformer, a mixer, and a condenser. The condenser includes a first casing, a second casing, a fluid mixture flow portion, and a gas-liquid separator. The first casing extends from a bottom portion side of the condenser to an upper portion side of the condenser opposite to the bottom portion side to serve as an outer shell of a main body of the condenser. The second casing is disposed inside the first casing and extends from the bottom portion side to the upper portion side so that a prescribed space is provided between an inner surface of the first casing and an outer surface of the second casing. The fluid mixture flow portion is provided in the prescribed space so that a gas-liquid fluid mixture passes through the fluid mixture flow portion.

### CROSS-REFERENCE TO RELATED APPLICATIONS

The present application claims priority under 35 U.S.C. §119 to Japanese Patent Application No. 2015-097541, filed May 12, 2015, entitled “Fuel Reforming Device.” The contents of this application are incorporated herein by reference in their entirety.

### BACKGROUND

1. Field

The present disclosure relates to a fuel reforming device.

2. Description of the Related Art

As is well known, antiknock quality is one of the important properties of a fuel for gasoline engines. Generally, the antiknock quality is rated by octane number. Recent high-compression ratio engines require a fuel with a particularly high octane number.

One method to control engine knocking under the condition that the octane number of the fuel used is constant is to retard ignition timing. However, when the ignition timing is retarded, the thermal efficiency of the engine is reduced. There is therefore a demand for development of a technique for controlling knocking while high thermal efficiency is maintained even in high-compression ratio engines.

It has previously been known that, in order to increase the octane number of gasoline and reduce the amount of hazardous substances contained in engine exhaust gas, an appropriate amount of alcohol (methanol) is added to the gasoline without addition of hazardous substances such as lead (see, for example, U.S. Pat. No. 4,244,328).

In a technique according the present disclosure described later, a gas produced in a treatment process is processed and separated into a condensed phase and a gas phase. A general processing device for separating a produced gas into gas and liquid has already been proposed (see, for example, Japanese Patent No. 3,335,555).

U.S. Pat. No. 4,244,328 discloses that the amount of hazardous substances contained in the engine exhaust gas is reduced by mixing methanol with gasoline through an exhaust gas recirculation passage including a catalytic reactor and supplying the resultant gas mixture to the engine.

However, in the technique disclosed in U.S. Pat. No. 4,244,328, it is necessary to store methanol in advance in a tank different from the tank of gasoline.

In the foregoing circumstances, the present applicant has recently proposed a fuel reforming device that can transform gasoline composed mainly of hydrocarbons into alcohols on a vehicle (Japanese Patent Application No. 2013-240400).

This fuel reforming device proposed by the present applicant includes, in the following order from an upstream side: a mixer for mixing air and a fuel composed mainly of hydrocarbons and supplying the air-fuel mixture to a reformer; the reformer for reforming the fuel using the air to produce alcohols; and a condenser for separating the gas produced in the reformer into a condensed phase and a gas phase.

The reformer of this fuel reforming device utilizes the action of primary and secondary catalysts. Specifically, the primary catalyst facilitates abstraction of hydrogen atoms from the hydrocarbons in the fuel to produce alkyl radicals, and the secondary catalyst facilitates reduction of alkyl hydroperoxides produced from the alkyl radicals to produce alcohols.

Preferably, the condenser used for the above-described fuel reforming device has a simple structure that can be produced easily, is suitable for size reduction, and is easy to maintain.

A general device for gas-liquid separation is disclosed in, for example, Japanese Patent No. 3,335,555, as described above.

### SUMMARY

According to one aspect of the present invention, a fuel reforming device for reforming a fuel composed mainly of a hydrocarbon using air to produce an alcohol includes a reformer, a mixer, and a condenser. The reformer includes a reforming catalyst for facilitating production of the alcohol that is produced by reforming the fuel composed mainly of the hydrocarbon using the air. The mixer is disposed upstream of the reformer. The mixer mixes the fuel and the air and supplies the fuel and the air mixed together to the reformer. The condenser is disposed downstream of the reformer. The condenser separates a gas produced in the reformer into a gas phase and a condensed phase composed mainly of the reformed fuel. The condenser includes a first casing that extends from a bottom portion of the condenser to an upper portion thereof and serves as an outer shell of a main body of the condenser, a second casing disposed within the first casing so as to extend from the bottom portion of the condenser to the upper portion thereof with a prescribed space formed between an inner surface of the first casing and an outer surface of the second casing, a fluid mixture flow portion formed in the space between the first casing and the second casing, the fluid mixture flow portion allowing a gas-liquid fluid mixture to pass therethrough, and a gas-liquid separator disposed in an upper portion of the second casing.

According to another aspect of the present invention, a fuel reforming device includes a reformer, a mixer, and a condenser. The reformer includes a reforming catalyst to reform, using air, a fuel including a hydrocarbon to produce an alcohol. The mixer is disposed upstream of the reformer to mix the fuel and the air. Mixture of the fuel and the air is supplied to the reformer. The condenser is disposed downstream of the reformer to separate a gas produced in the reformer into a gas phase and a condensed phase including a reformed fuel. The condenser includes a first casing, a second casing, a fluid mixture flow portion, and a gas-liquid separator. The first casing extends from a bottom portion side of the condenser to an upper portion side of the condenser opposite to the bottom portion side to serve as an outer shell of a main body of the condenser. The second casing is disposed inside the first casing and extends from the bottom portion side to the upper portion side so that a prescribed space is provided between an inner surface of the first casing and an outer surface of the second casing. The fluid mixture flow portion is provided in the prescribed space so that a gas-liquid fluid mixture passes through the fluid mixture flow portion. The gas-liquid separator is disposed in the second casing on the upper portion side of the condenser.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings.

FIG. 1 is an illustration showing the configuration of a fuel reforming device according to one embodiment of the present disclosure.

FIG. 2 is a side cross-sectional view of a condenser of the fuel reforming device in FIG. 1.

FIG. 3 is an exploded perspective view of the condenser of the fuel reforming device in FIG. 1.

FIG. 4A is a transverse cross-sectional view of one embodiment of the condenser of the fuel reforming device in FIG. 1.

FIG. 4B is a transverse cross-sectional view of another embodiment of the condenser.

### DESCRIPTION OF THE EMBODIMENTS

The embodiments will now be described with reference to the accompanying drawings, wherein like reference numerals designate corresponding or identical elements throughout the various drawings.

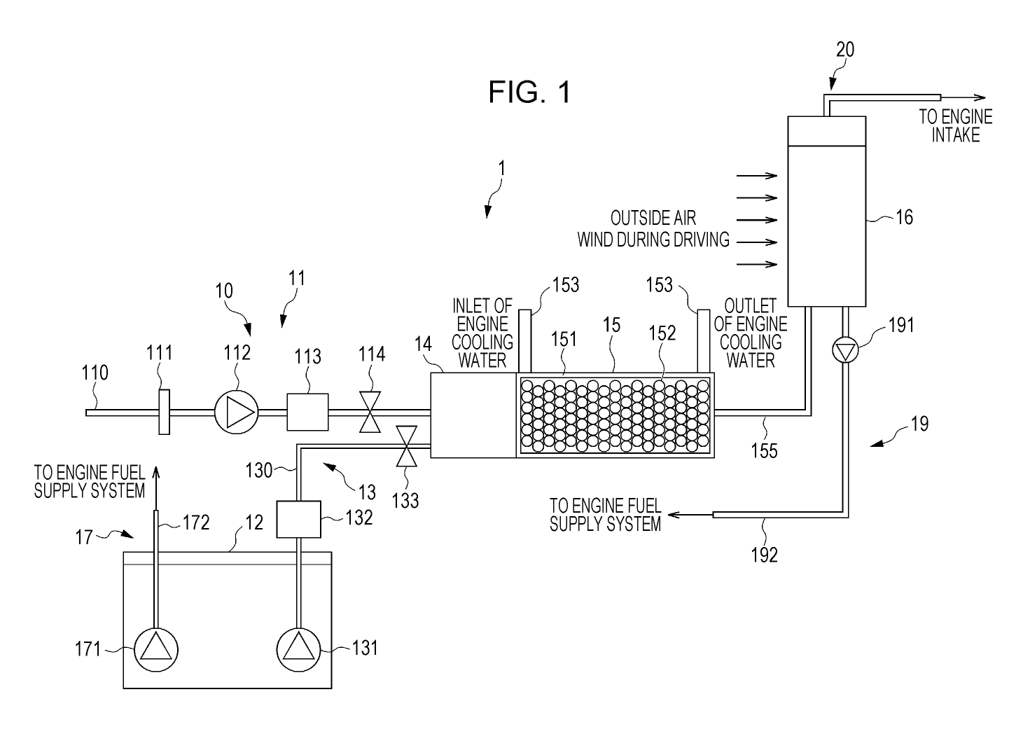

FIG. 1 is an illustration showing the configuration of a fuel reforming device 1 according to one embodiment of the present disclosure. The fuel reforming device 1 in the present embodiment is mounted on an unillustrated vehicle. The fuel reforming device 1 reforms hydrocarbons contained in a fuel into alcohols on the vehicle in response to a request from an unillustrated engine and supplies the produced alcohols to the engine.

In the fuel reforming device 1 in the present embodiment, gasoline is used as the fuel, and air is used as an oxidant. Specifically, in the fuel reforming device 1 in the present embodiment, an oxidation reaction with oxygen in air is used to reform the gasoline, so that the gasoline can be reformed at a lower temperature under milder conditions than those when the gasoline is reformed by, for example, a decomposition reaction. Therefore, the fuel reforming device 1 can have a simplified system configuration and is suitable for on-demand operation on the vehicle.

As shown in FIG. 1, the fuel reforming device 1 according to the present embodiment is configured to include an air introduction unit 11, a fuel tank 12, a fuel introduction unit 13, a mixer 14, a reformer 15, a condenser 16, a fuel supply unit 17, a reformed fuel supply unit 19, and a gas phase supply unit 20. As described later, in this fuel reforming device 1, the condenser 16 has a prescribed portion that forms a fuel tank for storing the reformed fuel. The reformed fuel is supplied from the condenser 16 (from its fuel tank) to an engine fuel supply system through a reformed fuel tube 192 by a reformed fuel pump 191.

The air introduction unit 11 is disposed upstream of the mixer 14 described later and introduces air serving as an oxidant into the mixer 14.

The air introduction unit 11 includes an air filter 111, an air pump 112, an air flow meter 113, and an air valve 114 that are disposed in this order from an upstream side of an air introduction tube 110.

When the air pump 112 is actuated, the air introduction unit 11 takes in air from the outside through the air filter 111. When the air valve 114 is opened, the air introduction unit 11 introduces the air taken in into the mixer 14.

The opening of the air valve 114 is controlled by an unillustrated electronic control unit (hereinafter referred to as an “ECU”) according to the flow rate of the air detected by the air flow meter 113, and the amount of air introduced into the mixer 14 is thereby controlled.

The fuel supply unit 17 includes a fuel pump 171, a fuel supply tube 172, and an unillustrated injector. When the fuel pump 171 is actuated, the fuel supply unit 17 supplies gasoline stored in the fuel tank 12 to a cylinder or an intake port of the unillustrated engine through the fuel supply tube 172 and the injector.

The amount of gasoline supplied to the engine is controlled by adjusting the injection amount of the injector by the ECU.

The fuel introduction unit 13 is disposed upstream of the mixer 14 described later and supplies the gasoline used as the fuel to the mixer 14.

The fuel introduction unit 13 includes a reforming pump 131, a fuel flow meter 132, and a fuel valve 133 that are disposed in this order from an upstream side of a fuel introduction tube 130.

When the reforming pump 131 is actuated and the fuel valve 133 is opened, the fuel introduction unit 13 introduces the gasoline stored in the fuel tank 12 into the mixer 14.

The opening of the fuel valve 133 is controlled by the ECU according to the flow rate of the fuel detected by the fuel flow meter 132, and the amount of gasoline introduced into the mixer 14 is thereby controlled.

In the fuel reforming device 1, a supply unit 10 for supplying the air and the fuel to the mixer 14 is configured to include the air introduction unit 11 and fuel introduction unit 13 described above.

In the supply unit 10, the air introduction unit 11 and the fuel introduction unit 13 cooperate with each other under the control of the ECU, and the air and fuel supplied to the mixer 14 are controlled such that the ratio of the fuel is 22% by weight or more.

As a result of the adjustment of the amounts of air and fuel, the air and fuel are supplied to the mixer 14 such that the ratio of the fuel is 22% by weight or more. This ratio corresponds to a ratio in a fuel-rich region, i.e., is higher than the upper explosive limit. Therefore, the fear of occurrence of an excessively abrupt reaction is minimized, so that the transformation process of transforming the gasoline into alcohols is stabilized.

The mixer 14 may be configured such that, for example, a connection portion between the air introduction tube 110 and the mixer 14 is formed to have a small hole diameter so that small air bubbles are generated. The mixer 14 may be configured such that a strong flow of air forms a vortex. The mixer 14 may be configured such that a particulate or porous material is disposed thereinside. In this case, the particulate or porous material causes the flow of the air and fuel supplied from the supply unit 10 to be dispersed, changed, and rotated, so that the air and fuel are mixed uniformly. The mixer 14 may be configured to include a static mixer so that the air and fuel are mixed uniformly.

The mixer 14 includes an unillustrated heater, and the gasoline and the air are mixed while heated to a prescribed temperature to thereby produce a gasoline-air mixture.

The reformer 15 disposed downstream of the mixer 14 described above reforms hydrocarbons, which are the main components of the gasoline in the gasoline-air mixture supplied from the mixer 14, using the air in the gasoline-air mixture to produce alcohols. Specifically, the reformer 15 may be any of a plug flow reactor and a completely mixed reactor.

In the plug flow reactor, a plug of the gasoline-air mixture introduced from the mixer 14 is not mixed with other plugs of the gasoline-air mixture supplied before and after the above plug within the reactor. The gasoline-air mixture flows as if it is pressed by a piston, is reformed, and then discharged. In the plug flow reactor, the composition of the fluid discharged from the reactor is different from the composition of the fluid inside the reactor. One characteristic of the plug flow reactor is that variations in residence time of the gasoline-air mixture within the reactor are small.

In the completely mixed reactor, the gasoline-air mixture introduced from the mixer 14 is mixed uniformly with reactants in the reformer and reformed. In the completely mixed reactor, the composition of the fluid discharged from the reactor is the same as the composition of the fluid inside the reactor. One characteristic of the completely mixed reactor is that variations in residence time of the gasoline-air mixture within the reactor are large.

In the fuel reforming device 1 in FIG. 1, a temperature sensor (not shown) and a cooling unit 153 for cooling the reformer 15 are disposed in the reformer 15. The cooling unit 153 is controlled by the ECU according to the temperature detected by the temperature sensor and supplies engine cooling water to the reformer 15 to cool the reformer 15.

The temperature of the engine cooling water is preferably 70° C. to 100° C. If the temperature of the engine cooling water is lower than 70° C., the rate of the reforming reaction is low. If the temperature of the engine cooling water exceeds 100° C., this engine cooling water is difficult to use. When the reforming reaction proceeds, the temperature inside the reformer 15 becomes high. In this case, the cooling unit 153 cools the reformer 15 with the engine cooling water. In contrast, in the early stage of the reforming reaction, i.e., when the temperature inside the reformer 15 is low, the cooling unit 153 functions to heat the reformer 15 with the engine cooling water.

The reformer 15 includes a reforming catalyst 152 that allows the hydrocarbons contained in the gasoline as main components to be reformed using the air serving as the oxidant to thereby produce alcohols. Specifically, the reformer 15 includes a cylindrical casing 151 and the reforming catalyst 152 in solid form filled into the casing 151.

The reforming catalyst 152 in solid form is configured to include a porous carrier in the form of small balls and primary and secondary catalysts supported on the surface of the porous carrier. A uniform mixture of the primary and secondary catalysts is supported on the surface of the porous carrier in the form of small balls. In the reforming catalyst 152 in the present embodiment, since the porous carrier has the form of small balls, the surface area of the primary and secondary catalysts supported on the surface of the porous carrier is large, so that the area of contact with the gasoline used as the fuel and the air used as the oxidant is large.

The porous carrier in the form of small balls is, for example, silica beads, alumina beads, or silica-alumina beads. Of these, silica beads are used preferably. The particle diameter of the porous carrier is preferably 3 μm to 500 μm.

The primary catalyst functions to facilitate abstraction of hydrogen atoms from the hydrocarbons in the gasoline to produce alkyl radicals. Specifically, the primary catalyst used is an N-hydroxyimido group-containing compound. Particularly, N-hydroxyphthalimide (hereinafter referred to as “NHPI”) and derivatives thereof have a strong catalytic action.

The secondary catalyst has an ability to reduce alkyl hydroperoxides produced from the alkyl radicals to produce alcohols. Specifically, the secondary catalyst used is a transition metal compound. Particularly, a compound selected from the group consisting of cobalt compounds, manganese compounds, and copper compounds is used preferably. Examples of the cobalt compound include cobalt(II) acetate. Examples of the manganese compound include manganese(II) acetate, and examples of the copper compound include copper(I) chloride.

Any known method such as an impregnation method can be used to support the primary and secondary catalysts described above on the porous carrier. For example, a slurry containing the primary and secondary catalysts in a prescribed ratio is prepared, and then the porous carrier in the form of small balls is immersed in the prepared slurry. Then the porous carrier is removed from the slurry. The excess slurry adhering to the surface of the porous carrier is removed, and the porous carrier is dried under prescribed conditions. A reforming catalyst 152 in which the primary and secondary catalysts are supported uniformly on the surface of the porous carrier is thereby obtained.

Next, the reforming reaction that proceeds in the reformer 15 will be described in detail.

In the reforming reaction in the present embodiment, a hydrogen abstraction reaction first proceeds. In the hydrogen abstraction reaction, hydrogen atoms are abstracted from the hydrocarbons in the gasoline, and alkyl radicals are thereby produced, as shown in the following reaction formula (1). The hydrogen abstraction reaction proceeds by the action of the primary catalyst, radicals, oxygen molecules, etc.

RH→R. reaction formula (1)

[In reaction formula (1), RH represents a hydrocarbon, and R. represents an alkyl radical.]

Then the alkyl radicals produced by the hydrogen abstraction reaction are bonded to oxygen molecules to form alkylperoxy radicals, as shown in the following reaction formula (2).

R.+O₂→ROO. reaction formula(2)

[In reaction formula (2), O₂ represents an oxygen molecule, and ROO. represents an alkylperoxy radical.]

Then the alkylperoxy radicals produced as shown in reaction formula (2) abstract hydrogen atoms from the hydrocarbons contained in the gasoline to produce alkyl hydroperoxides, as shown in the following reaction formula (3).

ROO.+RH→ROOH+R. reaction formula(3)

[In reaction formula (3), ROOH represents an alkyl hydroperoxide.]

Next, the alkyl hydroperoxides produced as shown in reaction formula (3) are reduced to alcohols by the action of the secondary catalyst, as shown in the following reaction formula (4).

ROOH→ROH reaction formula(4)

[In reaction formula (4), ROH represents an alcohol.]

At the same time, the alkyl hydroperoxides produced as shown in reaction formula (3) are decomposed into alkoxy radicals and hydroxyl radicals by the action of the secondary catalyst or heat, as shown in the following reaction formula (5).

ROOH→RO.+.OH reaction formula(5)

[In reaction formula (5), RO. represents an alkoxy radical, and .OH represents a hydroxyl radical.]

Next, the alkoxy radicals produced as shown in reaction formula (5) abstract hydrogen atoms from the hydrocarbons contained in the gasoline to produce alcohols.

RO.+RH→ROH+R. reaction formula(6)

As described above, the hydrocarbons contained in the gasoline as main components undergo oxidation reforming and are transformed into alcohols. More particularly, the hydrocarbons contained in the gasoline have 4 to 10 carbon atoms and are therefore transformed into alcohols having 4 to 10 carbon atoms. In the fuel reforming device 1 in the present embodiment, the octane number of the gasoline can be improved in the manner described above.

The condenser 16 is disposed downstream of the reformer 15 described above. In the condenser 16, the gas produced in the reformer 15 is separated into a gas phase and a condensed phase composed mainly of the reformed fuel. In the condenser 16, the produced gas supplied from the reformer 15 through a produced gas supply tube 155 is cooled to thereby separate the produced gas into the gas phase and the condensed phase composed mainly of the reformed fuel. The materials contained in the condensed phase include, in addition to the reformed fuel composed mainly of the alcohols, byproducts such as water, and the materials contained in the gas phase include nitrogen, oxygen, and other byproduct gas components.

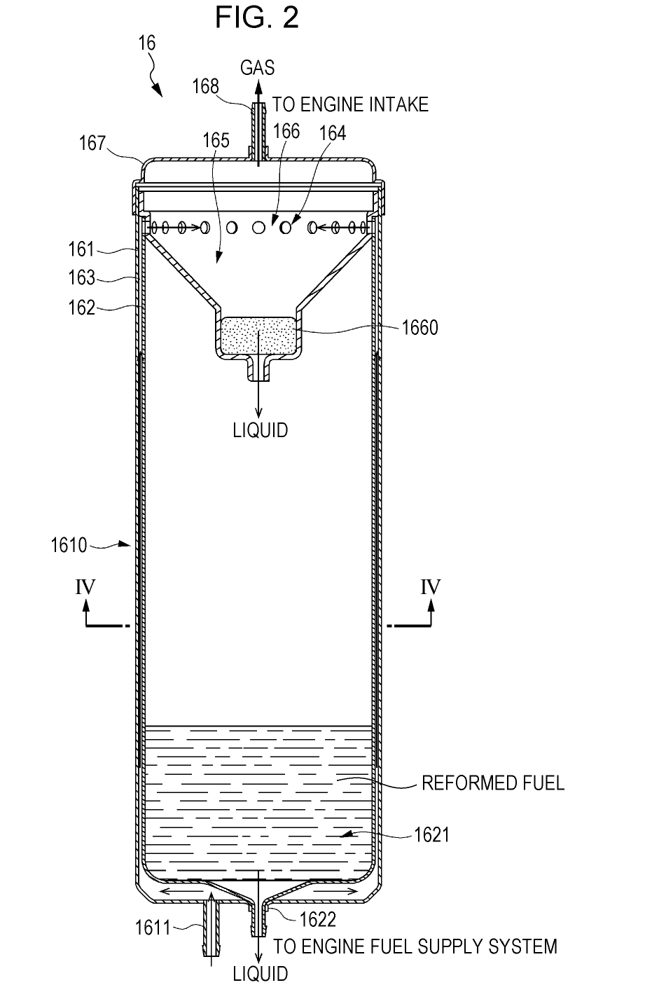

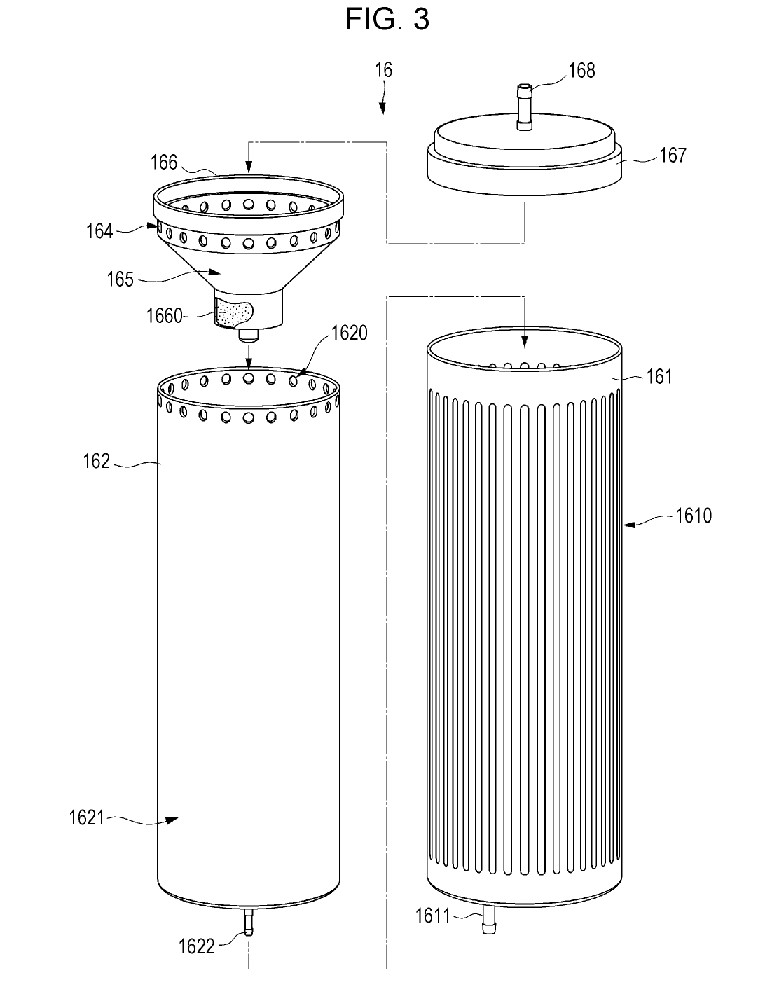

FIG. 2 is a side cross-sectional view of the condenser 16 of the fuel reforming device 1 in FIG. 1, and FIG. 3 is an exploded perspective view of the condenser 16. In FIGS. 2 and 3, the same parts are denoted by the same reference numerals.

In FIGS. 2 and 3, the condenser 16 includes: a tubular first casing 161 that extends from a bottom portion of the condenser 16 to its upper portion and serves as an outer shell of the main body; and a tubular second casing 162 that is disposed within the first casing 161 so as to extend from the bottom portion to the upper portion with a prescribed space formed between the outer surface of the tubular second casing 162 and the inner surface of the first casing 161.

As can be readily seen from FIG. 2, the first casing 161 and the second casing 162 form a double container, and the space between the first casing 161 serving as the outer container of the double container and the second casing 162 serving as the inner container forms a fluid mixture flow portion 163. Preferably, the width of the space forming the fluid mixture flow portion 163 is, for example, 1 mm or less.

A gas-liquid separator 166 is disposed in an upper portion of the double container, i.e., an upper portion of the second casing 162. The gas-liquid separator 166 includes: fluid mixture inlets 164 each having an opening through which a gas-liquid fluid mixture from the fluid mixture flow portion 163 flows; and a funnel-shaped liquid collector 165 that collects liquid phase materials resulting from gas-liquid separation of the gas-liquid fluid mixture flowing into the gas-liquid separator 166 through the fluid mixture inlets 164 and allows the collected liquid phase materials to flow downward toward the bottom portion of the second casing 162 by gravity.

A lid 167 is disposed so as to cover an upper opening of the double container, i.e., an upper opening of the gas-liquid separator 166. In the illustrated example, a skirt portion of the lid 167 is fitted externally to an upper edge portion of the first casing 161. The lid 167 has a discharge port 168 for discharging the gas phase materials resulting from gas-liquid separation in the gas-liquid separator 166.

The first casing 161 has a heat radiation portion 1610 on its outer surface. The heat radiation portion 1610 is configured such that, for example, a plurality of protruding ridges formed on the circumferential surface of the first casing 161 so as to extend vertically function as cooling fins, as shown in FIG. 3. These cooling fins facilitate the cooling effect of the ambient air.

The first casing 161 further includes, on its bottom, a fluid mixture introduction port 1611 in communication with the fluid mixture flow portion 163. In this example, the fluid mixture introduction port 1611 has a tubular shape. The produced gas supply tube 155 of the reformer 15 described above is connected to the fluid mixture introduction port 1611.

The second casing 162 has, in its upper portion, fluid mixture outlets 1620 corresponding to the fluid mixture inlets 164 of the gas-liquid separator 166. In the example in FIGS. 2 and 3, the diameter of a plurality of circular openings of the fluid mixture inlets 164 of the gas-liquid separator 166 is equal to the diameter of a plurality of circular openings of the fluid mixture outlets 1620 in the upper portion of the second casing 162. The second casing 162 and the gas-liquid separator 166 are positioned and combined such that the openings of the second casing 162 are matched and aligned with the openings of the gas-liquid separator 166.

A bottom portion of the second casing 162 forms a reformed fuel tank 1621 for storing the reformed fuel. A reformed fuel discharge port 1622 for discharging the reformed fuel to the outside is disposed in the bottom of the reformed fuel tank 1621 so as to pierce the bottom of the first casing 161. In this example, the reformed fuel discharge port 1622 has a tubular shape.

The second casing 162 may be formed to have a tapered overall shape such that its diameter decreases toward the bottom portion. In this configuration, the second casing 162 can be easily fitted into the first casing 161 during production, and this is well suitable for mass production.

As described above, in the present embodiment, a part of the second casing 162 forms the reformed fuel tank 1621. Therefore, it is unnecessary to provide a separate reformed fuel tank. In addition, since the reformed fuel discharge port 1622 for discharging the reformed fuel to the outside is disposed in the bottom of the reformed fuel tank so as to pierce the bottom of the first casing 161, a small and simple overall configuration is achieved.

A filter 1660 for preventing solid phase materials from passing through the gas-liquid separator 166 is disposed in a lower portion of the gas-liquid separator 166.

For example, a nonwoven fabric or a metal mesh such as a stainless steel mesh can be used for the filter 1660. The filter 1660 can prevent unnecessary solid phase materials from being mixed into the liquid phase material (reformed fuel).

In the condenser 16 described above, even after the first casing 161, the second casing 162, the gas-liquid separator 166, and the lid 167 are assembled, they can be separated from each other using a prescribed procedure. Therefore, the condenser 16 is easy to maintain.

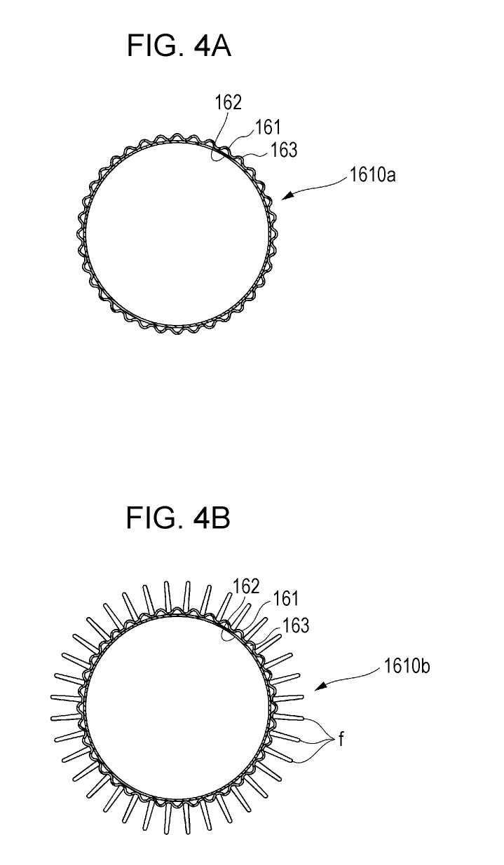

FIGS. 4A and 4B are transverse cross-sectional views of the condenser 16 in FIG. 1 taken along line IV-IV in FIG. 2.

FIG. 4A is a transverse cross-sectional view of one embodiment of the condenser 16, and FIG. 4B is a transverse cross-sectional view of another embodiment.

As described above with reference to FIG. 3, the space between the outer and inner casings 161 and 162, i.e., the first casing 161 and the second casing 162, of the double container serves as the fluid mixture flow portion 163, and the first casing 161 serving as the outer container has, on its outer surface, the heat radiation portion 1610. The heat radiation portion 1610 is configured such that, for example, the plurality of protruding ridges formed on the circumferential surface of the first casing 161 so as to extend vertically function as air-cooling type cooling fins, as shown in FIG. 3.

In the condenser 16 in the embodiment in FIG. 4A, a heat radiation portion 1610a of the first casing 161 is such that the first casing 161 itself has a circumferentially periodic wavy shape, as viewed in transverse cross section. The first casing 161 is in periodic contact with the second casing 162 serving as the inner container of the double container. Specifically, the heat radiation portion 1610a is in contact with the second casing at each of concave portions as viewed in cross section.

Therefore, the heat radiation effect of the heat radiation portion 1610a extends to the second casing 162, and a good cooling effect can be obtained in the fluid mixture flow portion 163 formed between the first casing 161 and the second casing 162.

In the condenser 16 in the embodiment in FIG. 4B, as in the embodiment in FIG. 4A, a heat radiation portion 1610b of the first casing 161 is such that the first casing 161 itself has a circumferentially periodic wavy shape as viewed in transverse cross section, and the first casing 161 is in partial contact with the second casing 162 serving as the inner container of the double container for every period.

In the heat radiation portion 1610b of the first casing 161 in the embodiment in FIG. 4B, plate-shaped fins f having a prescribed radial height as viewed in transverse cross section are disposed on the contact portions of the heat radiation portion 1610b that are in periodic contact with the second casing 162.

In the embodiment in FIG. 4B, since the heat radiation effect of the fins f is added, the cooling effect in the fluid mixture flow portion 163 formed between the first casing 161 and the second casing 162 is improved.

The condenser 16 described above may be disposed, for example, in a position close a radiator so that the heat radiation portion 1610 (1610a, 1610b) is easily exposed to the outside air such as wind during driving of the vehicle. Alternatively, the condenser 16 may be disposed in the trunk of a vehicle, and wind from an air conditioner may be supplied to the heat radiation portion 1610 (1610a, 1610b).

The fuel reforming device 1 in the present embodiment having the configuration described above is controlled by the ECU and operates as follows.

When a determination is made, based on the operating condition of the engine, that it is necessary to reform the gasoline, a determination is made as to whether or not the temperature of the engine cooling water is equal to or higher than a prescribed temperature. Immediately after the start of the engine, the temperature of the engine cooling water is lower than the prescribed temperature. In this case, the reformed fuel stored in the reformed fuel tank 1621 of the condenser 16 during the previous reforming operation is supplied to an intake port of the engine by the reformed fuel pump 191.

When the temperature of the engine cooling water is equal to or higher than the prescribed temperature, the fuel valve 133 and the air valve 114 are opened. Next, the gasoline is pumped from the fuel tank 12 by the reforming pump 131 and introduced into the mixer 14. Simultaneously, air passing through the air filter 111 is introduced into the mixer 14 by the air pump 112.

In the fuel reforming device 1 in the present embodiment, the air introduction unit 11 and the fuel introduction unit 13 in the supply unit 10 cooperate with each other under the control of the ECU, and the air and fuel supplied to the mixer 14 are controlled such that the ratio of the fuel (gasoline) is 22% by weight or more.

To obtain a desirable and appropriate reforming reaction time, the opening of the fuel valve 133 and the opening of the air valve 114 are feedback-controlled according to the gasoline flow rate monitored by the fuel flow meter 132 and the air flow rate monitored by the air flow meter 113 under the control of the ECU.

Next, the gasoline and air introduced into the mixer 14 are uniformly mixed while heated to a prescribed temperature to obtain a gasoline-air mixture, and the gasoline-air mixture is supplied to the reformer 15. The hydrocarbons, which are main component of the gasoline in the gasoline-air mixture supplied to the reformer 15, undergo the reactions in the above-described reaction formulas (1) to (6) by the action of the reforming catalyst 152 and are thereby transformed into alcohols. In this case, the supply of the engine cooling water is controlled according to the temperature monitored by the temperature sensor. The temperature inside the reformer 15 is thereby maintained at a desirable appropriate temperature.

Next, the gas produced in the reformer 15 is separated into a condensed phase and a gas phase in the condenser 16. The separated condensed phase is composed mainly of alcohols used as the reformed fuel, and the reformed fuel is stored in the reformed fuel tank 1621 of the condenser 16. The reformed fuel in the reformed fuel tank 1621 is supplied to the intake port of the engine by the reformed fuel pump 191. The separated gas phase materials are introduced into the intake port of the engine through the gas phase supply unit 20 and used for combustion in a cylinder of the engine.

When a determination is made, based on the operating condition of the engine, that it is unnecessary to reform the gasoline, the air pump 112 is stopped, and then the air valve 114 is closed, so that the supply of air to the mixer 14 is stopped. Then, after the reformer 15 is filled with the gasoline and all the air has escaped, the reforming pump 131 is stopped, and the fuel valve 133 is closed, so that the supply of the gasoline to the mixer 14 is stopped. This can prevent the reforming reaction from proceeding due to oxygen remaining in the reformer 15 when the system is suspended.

Next, the operation of the condenser 16 described with reference to FIGS. 2, 3, 4A, and 4B will be described in more detail.

The produced gas from the reformer 15 in FIG. 1 is supplied to the fluid mixture introduction port 1611 in the bottom of the condenser 16 through the produced gas supply tube 155. Specifically, the produced gas from the reformer 15 is supplied as a gas-liquid mixture to the condenser 16 from the bottom of the condenser 16. The supplied gas-liquid mixture spreads into a bottom space of the double container composed of the first casing 161 and the second casing 162 and then flows upward in the fluid mixture flow portion 163, which is a narrow-width space formed over the entire side circumference of the double container.

The fluid mixture flowing upward in the fluid mixture flow portion 163 comes into contact with the first casing 161 serving as a side wall of the fluid mixture flow portion 163 and is cooled through the heat radiation portion 1610 of the first casing 161. In the above-described embodiment in which the heat radiation portion 1610 is in contact with the second casing 162, also the second casing 162 has a cooling effect.

The fluid mixture flows upward in the fluid mixture flow portion 163 while being cooled and reaches the vicinity of the upper end of the fluid mixture flow portion 163. Then the fluid mixture flows through the openings of the fluid mixture outlets 1620 in the upper portion of the second casing 162 and flows into the gas-liquid separator 166 through the plurality of openings of the fluid mixture inlets 164 of the gas-liquid separator 166 that are aligned with the openings of the fluid mixture outlets 1620.

The reformed fuel (fuel composed mainly of alcohols) which is the liquid phase (condensed phase) in the fluid mixture flowing into the gas-liquid separator 166 is collected by the liquid collector 165 of the gas-liquid separator 166, filtrated through the filter 1660, flows downward within the second casing 162 by gravity, and is stored in the reformed fuel tank 1621 disposed on the bottom portion of the second casing 162.

The stored reformed fuel is delivered to the fuel supply system of the engine (not shown) from the reformed fuel discharge port 1622 on the bottom of the reformed fuel tank 1621 through the reformed fuel supply unit 19 in FIG. 1 under the control of the ECU as needed.

The materials contained in the fluid mixture flowing into the gas-liquid separator 166 and forming the gas phase are discharged to the outside of the gas-liquid separator 166 from the discharge port 168 disposed in the lid 167 on the upper portion of the gas-liquid separator 166. The discharged gas phase materials are supplied to the intake port through a passage in the gas phase supply unit 20 in FIG. 1 and used for combustion in the cylinder of the engine, as described above.

The fuel reforming device 1 in the present embodiment has the following effects.

(1) In the fuel reforming device 1 in the present embodiment, the condenser 16 includes: the first casing 161 extending from the bottom portion of the condenser 16 to its upper portion and serving as the outer shell of the main body; and the second casing 162 inside the first casing 161. The first and second casings 161 and 162 together form the double container. The space between the first casing 161 serving as the outer container of the double container and the second casing 162 serving as the inner container forms the fluid mixture flow portion. The gas-liquid separator 166 is disposed in the upper portion of the double container.

In this fuel reforming device 1, the condenser 16 has a simple structure that can be produced easily. The gas-liquid fluid mixture is cooled by the ambient air through the first casing 161 serving as the outer container when the gas-liquid fluid mixture flows through the fluid mixture flow portion 163. Therefore, no separate cooler and no coolant are required, and the device can be reduced in size.

(2) In the fuel reforming device 1 in the present embodiment, the condenser 16 includes the lid 167 that is disposed on the gas-liquid separator 166 so as to cover its upper opening and has the discharge port 168 for the gas phase materials resulting from gas-liquid separation. Specifically, since the lid 167 has the discharge port 168 for the gas phase materials resulting from gas-liquid separation, the overall structure of the condenser 16 is simple.

(3) In the fuel reforming device 1 in the present embodiment, the gas-liquid separator 166 of the condenser 16 includes: the fluid mixture inlets 164 that allow the gas-liquid fluid mixture from the fluid mixture flow portion 163 to flow into the gas-liquid separator 166; and the liquid collector 165 for collecting the liquid phase materials resulting from gas-liquid separation of the gas-liquid fluid mixture flowing from the fluid mixture inlets 164 and then allowing the collected liquid phase materials to flow downward toward the bottom portion of the second casing 162 by gravity.

Therefore, also the structure of the gas-liquid separator 166 is simple, and the overall structure of the condenser 16 is simple.

(4) In the fuel reforming device 1 in the present embodiment, the first casing 161 of the condenser 16 has, on its outer surface, the heat radiation portion 1610.

The heat radiation portion 1610 facilitates cooling of the first casing 161 by the ambient air, and this provides a good cooling effect on the gas-liquid fluid mixture flowing through the fluid mixture flow portion 163 formed along the inner surface of the first casing 161.

(5) In the fuel reforming device 1 in the present embodiment, the first casing 161 of the condenser 16 has, on its bottom, the fluid mixture introduction port 1611 in communication with the fluid mixture flow portion 163.

Therefore, a fluid mixture introduction passage to the fluid mixture flow portion 163 serving also as a cooler is very simple, and the overall structure of the condenser 16 is simple.

(6) In the fuel reforming device 1 in the present embodiment, the second casing 162 of the condenser 16 has, in its upper portion, the fluid mixture outlets 1620 corresponding to the fluid mixture inlets 164 of the gas-liquid separator 166.

Therefore, the fluid mixture flows into the gas-liquid separator 166 through the fluid mixture inlets 164 immediately from the fluid mixture outlets 1620, and the overall structure of the condenser 16 is simple.

(7) In the fuel reforming device 1 in the present embodiment, the bottom portion of the second casing 162 of the condenser 16 forms the reformed fuel tank 1621 for storing the reformed fuel, and the reformed fuel discharge port 1622 for discharging the reformed fuel to the outside of the condenser 16 is disposed in the bottom of the reformed fuel tank 1621 so as to pierce the bottom of the first casing 161.

Therefore, it is unnecessary of provide a separate reformed fuel tank, and the overall structure of the condenser 16 is simple.

(8) In the fuel reforming device 1 in the present embodiment, the heat radiation portion 1610 of the first casing 161 of the condenser 16 is partially in contact with the second casing 162.

Therefore, the heat radiation effect of the heat radiation portion 1610 extends to the second casing 162, and a good cooling effect can be obtained in the fluid mixture flow portion 163 formed between the first casing 161 and the second casing 162.

(9) In the fuel reforming device 1 in the present embodiment, the first casing 161, the second casing 162, the gas-liquid separator 166, and the lid 167 in the condenser 16 can be separated from each other.

Therefore, the condenser 16 is easy to maintain, i.e., byproducts etc. adhering to the interior of the condenser 16 can be easily removed.

The present disclosure is not limited to the embodiments described above, and modifications, improvements, etc. that fall within the scope of the present disclosure are included in the present disclosure.

(1) A fuel reforming device for reforming a fuel composed mainly of a hydrocarbon using air to produce an alcohol (for example, a fuel reforming device 1 described later), the device including: a reformer (for example, a reformer 15 described later) including a reforming catalyst for facilitating production of the alcohol that is produced by reforming the fuel composed mainly of the hydrocarbon using the air; a mixer (for example, a mixer 14 described later) disposed upstream of the reformer, the mixer mixing the fuel and the air and supplying the fuel and the air mixed together to the reformer; and a condenser (for example, a condenser 16 described later) disposed downstream of the reformer, the condenser separating a gas produced in the reformer into a gas phase and a condensed phase composed mainly of the reformed fuel; wherein the condenser includes a first casing (for example, a first casing 161 described later) that extends from a bottom portion of the condenser to an upper portion thereof and serves as an outer shell of a main body of the condenser, a second casing (for example, a second casing 162 described later) disposed within the first casing so as to extend from the bottom portion of the condenser to the upper portion thereof with a prescribed space formed between an inner surface of the first casing and an outer surface of the second casing, a fluid mixture flow portion (for example, a fluid mixture flow portion 163 described later) formed in the space between the first casing and the second casing, the fluid mixture flow portion allowing a gas-liquid fluid mixture to pass therethrough, and a gas-liquid separator (for example, a gas-liquid separator 166 described later) disposed in an upper portion of the second casing.

In the fuel reforming device in (1) above, the mixer for mixing the fuel composed mainly of the hydrocarbon and the air and supplying the air-fuel mixture to the reformer, the reformer for reforming the fuel using the air to produce the alcohol, and the condenser for separating the gas produced in the reformer into the condensed phase and the gas phase are disposed in this order from an upstream side.

Particularly, in the condenser, the first casing extending from the bottom portion to the upper portion and serving as the outer shell of the main body and the second casing inside the first casing together form a double container. The space between the first casing serving as the outer container of the double container and the second casing serving as the inner container forms the fluid mixture flow portion. The gas-liquid separator is disposed in the upper portion of the double container.

In the fuel reforming device in (1) above, the condenser has a simple structure that can be produced easily. The gas-liquid fluid mixture is cooled by the ambient air through the first casing serving as the outer container when the gas-liquid fluid mixture flows through the fluid mixture flow portion. Therefore, no separate cooler and no coolant are required, and the device can be reduced in size. With this fuel reforming device, gasoline composed mainly of hydrocarbons can be transformed into alcohols on a vehicle.

(2) In the fuel reforming device in (1) above, the condenser may further include a lid (for example, a lid 167 described later) that is disposed on the gas-liquid separator so as to cover an upper opening thereof, the lid including a discharge port (for example, a discharge port 168 described later) for a gas phase material resulting from gas-liquid separation.

In the fuel reforming device in (2) above, particularly in the fuel reforming device in (1) above, the condenser includes the lid that covers the upper opening of the double container and has the discharge port for the gas phase material resulting from gas-liquid separation. Therefore, the overall structure of the condenser is simple.

(3) In the fuel reforming device in (1) or (2) above, the gas-liquid separator of the condenser may include: a fluid mixture inlet (for example, fluid mixture inlets 164 described later) that allows the gas-liquid fluid mixture from the fluid mixture flow portion to flow into the gas-liquid separator; and a liquid collector (for example, a liquid collector 165 described later) for collecting a liquid phase material resulting from gas-liquid separation of the gas-liquid fluid mixture flowing into the gas-liquid separator through the fluid mixture inlet and then allowing the collected liquid phase material to flow downward toward a bottom portion of the second casing by gravity.

In the fuel reforming device in (3) above, particularly in the fuel reforming device in (1) or (2) above, the structure of the gas-liquid separator is also simple, so that the overall structure of the condenser is simple.

(4) In the fuel reforming device in any of (1) to (3) above, the first casing of the condenser may have, on an outer surface of the first casing, a heat radiation portion (for example, a heat radiation portion 1610 described later).

In the fuel reforming device in (4) above, particularly in the fuel reforming device in any of (1) to (3) above, the heat radiation portion on the outer surface of the first casing serving as the outer container of the double container of the condenser facilitates cooling of the first casing by the ambient air, and this provides a good cooling effect on the gas-liquid fluid mixture flowing through the fluid mixture flow portion disposed along the inner surface of the first casing.

(5) In the fuel reforming device in any of (1) to (4) above, the first casing of the condenser may have, in a bottom of the first casing, a fluid mixture introduction port (for example, a fluid mixture introduction port 1611 described later) in communication with the fluid mixture flow portion.

In the fuel reforming device in (5) above, particularly in the fuel reforming device in any of (1) to (4) above, the first casing of the condenser has, in the bottom of the first casing, the fluid mixture introduction port in communication with the fluid mixture flow portion. Therefore, the overall structure of the condenser is simple.

(6) In the fuel reforming device in (3) above, the second casing of the condenser may have, in an upper portion of the second casing, a fluid mixture outlet (for example, fluid mixture outlets 1620 described later) corresponding to the fluid mixture inlet of the gas-liquid separator.

In the fuel reforming device in (6) above, particularly in the fuel reforming device in (3) above, the second casing of the condenser has, in the upper portion of the second casing, the fluid mixture outlet corresponding to the fluid mixture inlet of the gas-liquid separator. Therefore, the fluid mixture flows into the gas-liquid separator through the fluid mixture inlet immediately from the fluid mixture outlet, and the overall structure of the condenser is simple.

(7) In the fuel reforming device in any of (1) to (6) above, a bottom portion of the second casing of the condenser may form a reformed fuel tank (for example, a reformed fuel tank 1621 described later) for storing the reformed fuel, and the reformed fuel tank may have, in a bottom thereof, a reformed fuel discharge port (for example, a reformed fuel discharge port 1622 described later) for discharging the reformed fuel to the outside of the condenser, the reformed fuel discharge port being disposed so as to pierce a bottom of the first casing.

In the fuel reforming device in (7) above, particularly in the fuel reforming device in any of (1) to (6) above, the bottom portion of the second casing of the condenser forms the reformed fuel tank for storing the reformed fuel. Therefore, it is unnecessary to provide a separate reformed fuel tank. In addition, the reformed fuel discharge port for discharging the reformed fuel to the outside of the condenser is disposed in the bottom of the reformed fuel tank so as to pierce the bottom of the first casing. Therefore, the overall structure of the condenser is simple.

(8) In the fuel reforming device in any of (4) to (7) above, the first casing may be formed such that the heat radiation portion is partially in contact with the second casing (see, for example, heat radiation portions 1610a and 1610b in embodiments in FIGS. 4A and 4B described later).

In the fuel reforming device in (8) above, particularly in the fuel reforming device in any of (4) to (7) above, the heat radiation portion of the first casing of the condenser is partially in contact with the second casing. Therefore, the heat radiation effect of the heat radiation portion extends to the second casing, and a good cooling effect can be obtained in the fluid mixture flow portion formed between the first casing and the second casing.

(9) In the fuel reforming device in any of (2) to (8) above, the condenser may be configured such that the first casing, the second casing, the gas-liquid separator, and the lid are separable from each other.

In the fuel reforming device in (9) above, particularly in the fuel reforming device in any of (2) to (8) above, the condenser is configured such that the first casing, the second casing, the gas-liquid separator, and the lid are separable from each other (see, for example, the first casing 161, the second casing 162, the gas-liquid separator 166, and the lid 167 in FIG. 3 described later). Therefore, the condenser is easy to maintain.

Obviously, numerous modifications and variations of the present invention are possible in light of the above teachings. It is therefore to be understood that within the scope of the appended claims, the invention may be practiced otherwise than as specifically described herein.

What is claimed is:

1. A fuel reforming device for reforming a fuel composed mainly of a hydrocarbon using air to produce an alcohol, the device comprising:
a reformer including a reforming catalyst for facilitating production of the alcohol that is produced by reforming the fuel composed mainly of the hydrocarbon using the air;
a mixer disposed upstream of the reformer, the mixer mixing the fuel and the air and supplying the fuel and the air mixed together to the reformer; and
a condenser disposed downstream of the reformer, the condenser separating a gas produced in the reformer into a gas phase and a condensed phase composed mainly of the reformed fuel; wherein
the condenser includes
a first casing that extends from a bottom portion of the condenser to an upper portion thereof and serves as an outer shell of a main body of the condenser,
a second casing disposed within the first casing so as to extend from the bottom portion of the condenser to the upper portion thereof with a prescribed space formed between an inner surface of the first casing and an outer surface of the second casing,
a fluid mixture flow portion formed in the space between the first casing and the second casing, the fluid mixture flow portion allowing a gas-liquid fluid mixture to pass therethrough, and
a gas-liquid separator disposed in an upper portion of the second casing.

2. The fuel reforming device according to claim 1, wherein the condenser further includes a lid that is disposed on the gas-liquid separator so as to cover an upper opening thereof, the lid including a discharge port for a gas phase material resulting from gas-liquid separation.

3. The fuel reforming device according to claim 1, wherein the gas-liquid separator of the condenser includes:
a fluid mixture inlet that allows the gas-liquid fluid mixture from the fluid mixture flow portion to flow into the gas-liquid separator; and
a liquid collector for collecting a liquid phase material resulting from gas-liquid separation of the gas-liquid fluid mixture flowing into the gas-liquid separator through the fluid mixture inlet and then allowing the collected liquid phase material to flow downward toward a bottom portion of the second casing by gravity.

4. The fuel reforming device according to claim 1, wherein the first casing of the condenser has, on an outer surface of the first casing, a heat radiation portion.

5. The fuel reforming device according to claim 1, wherein the first casing of the condenser has, in a bottom of the first casing, a fluid mixture introduction port in communication with the fluid mixture flow portion.

6. The fuel reforming device according to claim 3, wherein the second casing of the condenser has, in an upper portion of the second casing, a fluid mixture outlet corresponding to the fluid mixture inlet of the gas-liquid separator.

7. The fuel reforming device according to claim 1, wherein a bottom portion of the second casing of the condenser forms a reformed fuel tank for storing the reformed fuel, and the reformed fuel tank has, in a bottom thereof, a reformed fuel discharge port for discharging the reformed fuel to the outside of the condenser, the reformed fuel discharge port being disposed so as to pierce a bottom of the first casing.

8. The fuel reforming device according to claim 4, wherein the first casing is formed such that the heat radiation portion is partially in contact with the second casing.

9. The fuel reforming device according to claim 2, wherein the condenser is configured such that the first casing, the second casing, the gas-liquid separator, and the lid are separable from each other.

10. A fuel reforming device comprising:
a reformer including a reforming catalyst to reform, using air, a fuel including a hydrocarbon to produce an alcohol;
a mixer disposed upstream of the reformer to mix the fuel and the air, mixture of the fuel and the air being supplied to the reformer; and
a condenser disposed downstream of the reformer to separate a gas produced in the reformer into a gas phase and a condensed phase including a reformed fuel, the condenser comprising:
a first casing extending from a bottom portion side of the condenser to an upper portion side of the condenser opposite to the bottom portion side to serve as an outer shell of a main body of the condenser;
a second casing disposed inside the first casing and extending from the bottom portion side to the upper portion side so that a prescribed space is provided between an inner surface of the first casing and an outer surface of the second casing;
a fluid mixture flow portion provided in the prescribed space so that a gas-liquid fluid mixture passes through the fluid mixture flow portion; and
a gas-liquid separator disposed in the second casing on the upper portion side of the condenser.

11. The fuel reforming device according to claim 10, wherein the condenser further includes a lid that is disposed on the gas-liquid separator so as to cover an upper opening of the gas-liquid separator opening toward the upper portion side of the condenser, the lid including a discharge port for a gas phase material resulting from gas-liquid separation.

12. The fuel reforming device according to claim 10, wherein the gas-liquid separator of the condenser includes:
a fluid mixture inlet through which the gas-liquid fluid mixture from the fluid mixture flow portion is to flow into the gas-liquid separator; and
a liquid collector to collect a liquid phase material resulting from gas-liquid separation of the gas-liquid fluid mixture flowing into the gas-liquid separator through the fluid mixture inlet so that the liquid phase material flows downward toward a bottom portion of the second casing by gravity.

13. The fuel reforming device according to claim 10, wherein the first casing of the condenser has, on an outer surface of the first casing, a heat radiation portion.

14. The fuel reforming device according to claim 10, wherein the first casing of the condenser has, in a bottom of the first casing, a fluid mixture introduction port in communication with the fluid mixture flow portion.

15. The fuel reforming device according to claim 12, wherein the second casing of the condenser has, in an upper portion of the second casing, a fluid mixture outlet corresponding to the fluid mixture inlet of the gas-liquid separator.

16. The fuel reforming device according to claim 10, wherein a bottom portion of the second casing of the condenser provides a reformed fuel tank to store the reformed fuel, and the reformed fuel tank has, in a bottom of the reformed fuel tank, a reformed fuel discharge port to discharge the reformed fuel to outside of the condenser, the reformed fuel discharge port being disposed so as to pierce a bottom portion of the first casing.

17. The fuel reforming device according to claim 13, wherein the first casing is provided such that the heat radiation portion is partially in contact with the second casing.

18. The fuel reforming device according to claim 11, wherein the condenser is configured such that the first casing, the second casing, the gas-liquid separator, and the lid are separable from each other.

19. The fuel reforming device according to claim 10, wherein a width of the prescribed space is 1 mm or less.

20. The fuel reforming device according to claim 13, wherein the heat radiation portion comprises protruding ridges provided on a circumferential surface of the first casing so as to extend from the upper portion side to the bottom portion side of the condenser.
